(19) United States
(12) Patent Application Publication
(10) Pub. No.: US 2011/0174898 A1
(43) Pub. Date: Jul. 21, 2011

(54) METHOD FOR CONTROLLING SOIL INSECTS

(75) Inventor: Xavier Peyron, Marseille (FR)

(73) Assignee: SBM Developpement, Eculy (FR)

(21) Appl. No.: 13/001,526

(22) PCT Filed: Jun. 24, 2009
(86) PCT No.: PCT/FR2009/000770

§ 371 Date: Apr. 1, 2011

(30) Foreign Application Priority Data
Jun. 25, 2008 (FR) 0803577

(51) Int. Cl. A61L 9/04; A01N 37/34; A01P 7/04; A01C 7/00; A01C 5/06

(52) U.S. Cl. 239/34; 514/520; 111/170

(57) ABSTRACT

The subject matter of the invention is the use, by burial in the soil, of a composition based on pyrethrum, with the exception of tefluthrin and of bifenthrin, and a method of controlling soil insects using said pyrethrum-based composition.

The present invention relates to the field of agriculture, and more particularly to the use of a composition based on pyrethrum, with the exception of tefluthrin and bifenthrin, in the soil with ploughing-in.

The term pesticide, derived from the word pest, denotes substances or preparations used for the prevention, control or eradication of organisms considered to be undesirable, whether they are plants, animals, fungi or bacteria. In common parlance the term pesticide is generally associated with the agricultural use of these substances, but the generic term also includes domestic and municipal uses, use for road maintenance etc.

In the present text, the term pesticide denotes either the active ingredient, i.e. responsible for the intended action, and the composition containing the active ingredient and sold to the user. In the field of agriculture, they are called phytopharmaceutical products (or plant protection products). There are basically three categories: herbicides (to control weeds), fungicides (to control fungi) and insecticides (to control insects). There are other products having action on rodents (rodenticides), and on snails and slugs (molluscicides).

Pesticides have been used in agriculture since ancient times. The use of sulphur appears to date back to 1000 years B.C., arsenic was recommended by Pliny and the arsenical products have been known in China since the XVI century; the insecticidal properties of tobacco and of roots of Derris and of Lonchocarpus were also noted around that era.

The more generalized use of pesticides followed advances in inorganic chemistry. In the XIX century, fungicidal treatments were based on copper sulphate (including the famous Bordeaux mixture) or based on mercury; insecticides such as copper arsenite, copper acetoarsenite, and lead arsenate also made their appearance. Pyrethrum, a powder obtained from flowers of the genus Chrysanthemum, was introduced as an insecticide in this same period.

Then, pesticides received a considerable boost with the development of organic chemistry even before World War II; and especially thereafter. It was in this period that a large number of organic compounds appeared.

In the 1950s, insecticides such as DDD and DDT were used in large quantities in preventive medicine for destroying the mosquito responsible for malaria and in agriculture for eradicating the Colorado beetle.

There has been considerable development in the use of these products in recent decades, making them almost indispensable for most agricultural practice, regardless of countries' levels of economic development. From 1945 to 1985, the consumption of pesticides doubled every ten years.

Among the pesticides, insecticides are the active ingredients or preparations having the property of killing insects, their larvae and/or their eggs. The general term insecticide also includes pesticides intended to control arthropods, which are not insects (e.g. mites, spiders or tics), as well as repellents.

There are various chemical classes of insecticides, which are related to their mode of action, which may be based on disturbance of the nervous system, of cellular respiration, the formation of the cuticle, or disturbance of moulting. These main classes are the organophosphorus compounds, the carbamates, the natural or synthetic pyrethroids, the organochlorine compounds and the benzoyl ureas.

Among the various uses of insecticides, the protection of seedlings against soil insects is still crucial for certain crops. The year 2006 marks an upsurge in damage caused by underground pests, for example grey fly, and click beetles. The latter were extremely prevalent in the autumn of 2005 to some extent everywhere and more particularly in the west of France. Only protection of seeds provides an effective response against these pests.

Crops of maize, beetroot, sunflower, potato and colza are still very liable to destruction by the click beetle, and the almost complete destruction of plots is sometimes observed in the absence of treatment. The click beetles constitute a family of insects that are particularly harmful to these crops, and their harmful character is even more pronounced as the larval forms of the click beetles can remain in the soil for very long periods of up to 5 years.

Although pesticides have made an enormous contribution to the management of food resources and improvement of public health (in particular for controlling disease-carrying insects), the other side of the coin soon appeared with the development of resistance in insects, sex change in certain batrachians and fertility problems in certain invertebrates.

Pollution with pesticides has been detected in all compartments of the environment: in river water and groundwater, in the air and in rain water. They are also found in fruit, vegetables, cereals and products of animal origin (eggs, milk, meat, fish etc.). They exist in their initial form but they may also be degraded, and are then called residues or metabolites.

Thus, although insecticides have been adopted, they still give rise to disquiet in particular because of the environmental problems connected with their misuse. We are therefore still trying to find an agent that can effectively control “pests” yet displays the minimum possible toxicity, or even no toxicity of all.

In most countries the marketing and use of insecticides are subject to prior authorization (approval or marketing authorization) by the competent national authority.

For many years, numerous plant protection products previously authorized (and therefore considered as effective and not presenting unacceptable risks) have been banned or are going to be banned from marketing and use, for example atrazine, lindane, and DDT.

Those that are still on the market offer the advantage that either they are less toxic, or they are highly active, and can thus be spread at low dose, which avoids not only the pollution mentioned above, but also problems of costs associated with spreading large amounts of insecticide.

However, the European overall programme of ecological reform of agriculture envisages banning, from 2008, nearly 400 products that are judged to be dangerous to human health, although they had been approved by the 1991 directive.

There is therefore a constant real need for active ingredients that are more effective and less harmful, for formulations that are more effective, or for methods of application optimizing the “contacting” of the pesticide with the plant and/or with the harmful animal.

At present, throughout the world there are nearly 100,000 commercial specialities authorized for sale, comprising about 800 different active ingredients. 15 to 20 new active ingredients are added each year.

The main types of formulation are as follows:

+Solid presentations:
wettable powders (WP): the active ingredient is finely ground (solid) or fixed (liquid) on an adsorbent or porous support (silica), Surfactants (dodecylbenzene sulphonate, lignosulphonate of Ca, Al or Na) and diluting fillers (kaolin, talc, chalk, aluminium and magnesium silicate or calcium carbonate) are added, as well as antiredeposition agents, antistatic or anti-foaming agents. Stabilizers (anti-oxygen and pH buffer) are included to make them compatible with other preparations. These powders must be dispersed in water at the moment of use;
water dispersible granules (WG): granules obtained by agglomeration with a little water, active ingredient, filler and binders and dispersants, followed by drying. These granules must be dispersed in water at the moment of use;
microgranules (MG): granules obtained by agglomeration of active ingredient, filler and binders and dispersants, followed by drying. The microgranules are formulated to be used dry and are ready to use. They are calibrated and have a uniform size to permit even spreading of the active ingredient. The filler is a mineral filler;
granular bait (GB): granules obtained by agglomeration of active ingredient, filler and binders and dispersants, followed by drying. Granular bait is formulated ready to use. It is calibrated and has a uniform size to permit even spreading of the active ingredient. The filler is a flour (wheat, durum wheat, etc.).

+Liquid presentations:
soluble concentrates (SL): this is a solution of active ingredient to be diluted in water, with added surfactants;
concentrated suspensions (SC): the solid active ingredients, insoluble in water, are in concentrated suspension in water, in the presence of wetting agents, dispersants, thickeners (xanthan gum, bentonite, silica) or anti-redeposition agent, antigelling agent (ethylene glycol, urea), antifoaming agents and sometimes bactericides (1,2-benzisothiazol-3(2H)-one, methanal or formol). These preparations are diluted in water at the moment of use;
emulsifiable concentrates (EC): a concentrated solution of the active ingredients is prepared in an organic solvent and emulsifiers are added for stabilizing the emulsions obtained at the moment of use by dilution in water;
concentrated emulsions (EW): the active ingredient is dissolved in an organic solvent. The solution, to which emulsifiers are added, is dispersed in a large amount of water. This presentation is less toxic and less flammable than the emulsifiable concentrates. It is diluted in water at the moment of use.

The pesticides can be sold as powders or as concentrates to be diluted with water, as aerosol, granules or bait. They are applied in various ways; they can be sprayed from an aircraft or from a sprayer optionally drawn by a tractor, dissolved in water for irrigation, ploughed into the soil, spread as granules or as pellets on the soil near the plants, applied as treatment of seeds, inserted in the collar of cattle or presented in the form of bait.

With some exceptions, these substances are not used pure: they are either diluted in water or in another solvent (hydrocarbon), or mixed with an inert solid material (clay, talc, charcoal, etc.) and presented in the form of powder or granules, or combined with a foodstuff serving as bait (wheat for rodents, bran for snails and slugs).

One way to try to protect seeds is to spread insecticide granules or bait on the surface, at the time of sowing. However, the existing methods of application do not provide optimum protection. In fact, they are not placed all around the seed, and harmful insects can reach the seed and damage it. Moreover, these formulas are not very active at low doses.

To overcome this drawback, certain products are applied directly on seeds, for example using a process called coating, by which the product is made to adhere to the seed. These solutions are not suitable for all types of seeds, nor for every type of insecticide, nor against all types of soil pests.

Another method of application is to put insecticide granules or bait at the bottom of the seed furrow, at the time of sowing. The advantage of this method of application is that there is improved contact of the product with the seed since the product is closer to the seed, in comparison with application by surface spreading. However, this method of application can only be considered for products that act by the vapour effect, i.e. which, once applied in the soil, diffuse in the soil so that they act on a larger area of soil than the area represented by the surface area of the solid product itself.

There is therefore a need for a method of application of insecticidal compounds, particularly of insecticidal compounds without a vapour effect, that are more effective for protecting seeds against harmful insects, at the time of sowing.

One of the purposes of the present invention is to provide a novel method of application for insecticide compositions whose active ingredient is one or more compounds of the pyrethroid class, with the exception of tefluthrin and bifenthrin, or their isomers, which overcome the problems of the solutions of the prior art.

In fact the pyrethroids represent one of the rare families of insecticidal compounds that still have marketing authorization, and in particular for protecting seeds.

By “pyrethroid” is meant, according to the invention, unless stated otherwise, both natural pyrethrum and the synthetic pyrethroids, cypermethrin in particular.

The pyrethrum, or Dalmatian pyrethrum, is a hardy herbaceous plant of the family Compositae. It is a species that originated from south-east Europe (Croatia, Montenegro, Albania). It is a hardy plant that grows to a height of 40 to 60 cm, growing in clumps with numerous stems each bearing a terminal capitulum. It has been spread widely by cultivation, in particular in Europe (Italy, Spain), to Japan, North Africa, Kenya, and Rwanda. This plant is cultivated for its flowers, from which an insecticidal powder is extracted.

The term “pyrethrum” denotes the powder made from the dried flowers of the chrysanthemum whereas the term “pyrethrin(s)” denotes the six insecticidal compounds contained naturally in this powder. These six pyrethrins constitute from 0.9 to 1.3% of the dried flowers.

Commercially, purification of the pyrethrins is generally attempted. After initial extraction, refining is carried out to remove the resins, waxes and allergens. The extract is then used in the preparation of various insecticides. This complex industrial process optimizes the efficacy of the product. Adjuvants can be added, to increase the efficacy of pyrethrum, or surfactants, or antioxidants.

As well as these natural insecticidal compounds, there is a whole range of synthetic pyrethroids, which are inexpensive and very effective. The synthetic pyrethroids are called “third generation” insecticides; they were derived from natural pyrethrums, while endeavouring to increase their toxicity and their photostability. Endowed with considerable toxicity and acting by contact, they kill insects almost instantaneously by neurotoxic shock, so that they can be used at very low doses (10 to 40 g of active ingredient per hectare). They kill insects by blocking the functioning of the sodium channels, which are indispensable to the transmission of nerve impulses.

Reputed to be of low toxicity for humans, they are attributed the highest safety factor (ratio of toxicity to insects to toxicity to mammals) among the chemical insecticides. Being very biodegradable, they do not persist in the soil, but these compounds are toxic to certain aquatic organisms and to aids to agriculture, such as bees.

Examples of synthetic pyrethroids are: bifenthrin, bioresmethrin, deltamethrin, depallethrin, etofenprox, fenpropathrin, cypermethrin, fenvalerate, esfenvalerate, cyfluthrin, alphametrine, betacypermethrin, tralomethrin, fluvalinate, permethrin, lambda-cyalothrin, flucythrinate, tefluthrin, tralomethrin, zetacypermethrin, betacyfluthrin.

The inventors had the idea of improving the usual method of application of formulations based on pyrethrum, with the exception of tefluthrin and bifenthrin. The inventors demonstrated that by applying pyrethrum-based compositions, with the exception of tefluthrin and bifenthrin, in the form of granules or microgranules, the efficacy of these compounds for the protection of seeds was increased considerably. In particular, using pyrethrums with ploughing in can constitute a barrier around the seed preventing pests reaching the latter. Thus, the active ingredient is in the best possible conditions as it is close to the seed and can even prevent its biochemical target reaching the seed. With this aim, their chances of contact with the target pest are increased by the formation of this barrier.

Unexpectedly, the applicant in fact found that the effect of pyrethrum, in the form of granules or microgranules, was greatly increased when the latter are applied by ploughing in over the full width of the seed furrow in which the seed is placed, whether the crop is grown in furrows or in ridges. Applied in this way, these compounds form a barrier to soil insects, which can less easily attack and denature the seed, as well as the underground parts of the plant, once the seed has germinated and the seedling has started to grow. A greater effect of the pyrethrum derivatives is thus obtained when used in this way, in comparison with conventional means of application, especially when the insecticides are applied in liquid form.

The pyrethrums, being compounds that act by contact, and not by a vapour effect, are usually employed in a liquid formulation. They are not used formulated as granules or microgranules because applied conventionally, i.e. only at the bottom of the seed furrow, their effect is very localized and very limited.

The use of pyrethrum, with the exception of tefluthrin and bifenthrin, in the form of granules or microgranules, with ploughing-in over the full width of the seed furrow is advantageous because the product, being present in the soil all around the seed, can have an effect all around the seed. Applied in this way, the efficacy of the products is increased relative to the liquid formulations.

Another advantage is that the doses of product applied can therefore be reduced, with a consequent decrease in their toxicity, both for the environment and for the person applying the product.

Yet another advantage is that the use of microgranules, because they are not captured by the colloids in the soil (such as clay), pose less environmental risk. In fact, products used in liquid form, for example, can be captured by these soil colloids. There is then a risk of runoff under the action of heavy rain, which carries away the colloids and therefore the product to the rivers. Moreover, their sequestration makes them less bioavailable (contact between the product and the pest is disturbed by the layers of colloid). This is not the case with microgranules.

It is on the basis of these results that the inventors propose the use, with ploughing into the soil, of a composition comprising at least pyrethrum, with the exception of tefluthrin and bifenthrin, in the form of granules or microgranules, preferably in the form of microgranules.

Hereinafter, unless stated otherwise, the term “pyrethrum” denotes pyrethrum per se, pyrethrins and/or pyrethroids, whether they are natural or synthetic, as well as all their isomers or mixtures of isomers.

Thus, in its first subject, the invention relates to the use, with ploughing into the soil, of a composition comprising at least pyrethrum with the exception of tefluthrin and bifenthrin, in the form of granules or microgranules.

According to a preferred embodiment, the invention relates to the use, with ploughing into the soil, of a composition comprising at least pyrethrum, with the exception of tefluthrin and bifenthrin, in the form of microgranules.

According to the invention, the synthetic pyrethrums are selected from bioresmethrin, deltamethrin, depallethrin, etofenprox, fenpropathrin, cypermethrin, fenvalerate, esfenvalerate, cyfluthrin, alphametrine, betacypermethrin, tralomethrin, fluvalinate, permethrin, lambda-cyalothrin, flucythrinate, tralomethrin, zetacypermethrin, betacyfluthrin. Among the compounds described above, cypermethrin and natural pyrethrum are used more particularly.

According to the invention, the pyrethrums can be used alone or mixed either with one another or with other pesticide compounds.

When, according to the invention, the composition is to be in the form of microgranules, the latter can have a size between 0.1 and 1 mm in diameter, preferably between 0.25 and 0.8 mm.

The apparent density of the microgranules is important as it determines good spreading of the latter.

Thus, according to the invention, when the composition is to be in the form of microgranules, the latter can have an apparent density between 1 and 2, preferably between 1.35 and 1.65.

By “apparent density” is meant the ratio of the weight of the sample to the volume of said sample (ad=W/V), (YORO G. and GODO G., Call, ORSTOM, set: Pédol., Vol. XXV, No. 4, 1989-1990: 423-429).

The number of grains per gram of microgranules is important as it determines the good efficacy of the latter. In fact, with increase in the number of grains per gram of, microgranules, there is a greater chance of contact between the microgranules and the pest, and therefore better efficacy of the microgranules.

Thus, when, according to the invention, the composition is to be in the form of microgranules, the latter can have a number of grains per gram between 1500 and 4000, preferably between 2200 and 3400.

According to the invention, the application dose of the compositions varies depending on the compound used.

By way of example, according to the invention, when the active ingredient is pyrethrum per se, the amount of active ingredient used in the composition can be in a proportion corresponding to 2 to 12 grams of active ingredient per kilogram of composition, preferably from 6 to 10 g/kg, and very preferably from 7 to 9 g/kg.

According to the invention, the composition can be used at a dose in the range from 2 to 20 kg/ha, preferably from 9 to 15 kg/ha, and very preferably of 12 kg/ha.

According to a preferred embodiment, the invention relates to the aforementioned use, in a method of protection of a furrow crop against soil insects, which comprises the following steps:
a) a furrow is made in the soil;
b) the seed is placed on the earth at the bottom of the seed furrow formed by the furrow;
c) then a composition comprising at least pyrethrum, with the exception of tefluthrin and bifenthrin, is spread over the full width of the seed furrow in which the seed has been placed; and
d) the furrow is covered with the earth prepared in step c).

According to a variant, the second and third steps of the method can be reversed.

According to yet another variant, the second and third steps can be simultaneous.

Thus, three orders of application are possible. Either the seed is put on the earth, then the pyrethrum-based preparation is applied, or vice versa. Another possibility is simultaneous application of seed and bait.

Preferably the pyrethrum-based preparation is deposited first, then the seed.

According to the method, during creation of the furrow, an excavation in the form of a half cylinder is formed in the soil. Application of the composition comprising at least one pyrethrum over the whole surface of the excavation, before or after or at the moment of depositing the seed, makes it possible, during closure of the furrow, to create an insecticide barrier all around the seed, the latter being enveloped in a network of insecticide, which protects it from the soil insects, which are unable to reach it.

The invention also applies to ridge crops. In this case, the composition based on pyrethrum, with the exception of tefluthrin and bifenthrin, is applied on a surface of the soil greater than the surface occupied by the seed, before or after or at the moment of depositing the seed, and when the ridge is formed, the seed is enclosed in earth mixed with the insecticide composition, all around it, which protects it effectively against insect pests in the soil. The seed is therefore completely surrounded by the formulation comprising the active ingredient.

The invention also relates to a method of protection of a ridge crop against soil insects, which comprises the following steps:
a) the seed is placed on the earth,
b) then a composition comprising at least pyrethrum, with the exception of tefluthrin and bifenthrin, is spread on an area equivalent to the largest circumference of the seed,
c), a ridge is formed by covering the seed placed in a) with the earth prepared in the preceding step.

According to a variant, the first and the second step of the method of growing in a ridge can be reversed.

According to another variant, the first and the second step of the method of growing in a ridge can be simultaneous.

Preferably according to the invention, the pyrethrum-based composition is deposited first, then the seed.

The present invention therefore relates to a method for protecting seeds that offers the following advantages:
the microgranulated compounds are well distributed in the seed furrow over the full profile of the furrow. Thus, during closure of the furrow or creation of the ridge, the active compound is distributed all around the seed. The seed is thus protected against soil insects. The latter have greater difficulty in reaching the seed.
the use of a composition based on pyrethrum, with the exception of tefluthrin and bifenthrin, with ploughing-in according to the invention is more effective closest to the seed, without being on the seed. The method according to the invention therefore optimizes protection of the seed without the drawbacks of coated seeds. It is understood that the insecticide, forming a barrier, kills a large number of soil insects trying to reach the seed. Because of this increased efficacy of the insecticide relative to the usual methods of application, the method is particularly advantageous in that it permits the use of lower doses of the active product,
the method according to the invention protects the seed, but also the underground parts of the plant following germination of the seed,
the farmer obtains a higher yield because fewer seeds are damaged,
the inventors have shown that the efficacy of the product is increased when using the method according to the invention, relative to a conventional method,
the method according to the invention can, moreover, be applied easily and quickly,
dispersion of insecticide in the environment is also limited. In fact, the method of application of the insecticide does not cause any spread or pollution on the surface of the soil, which avoids contamination of animals other than the animals living in the soil.

The method according to the invention, can be used for all agricultural crops (cereals, market garden plants, horticultural plants, etc.). It is to be understood that the seeds and then the plant emerging from the seed are protected by the method according to the invention. It is preferably used for crops of maize, colza, sorghum, sunflower, potato, beetroot, carrot, cereals, cabbages, beans, melon, tomato, ornamental plants or even tobacco. Even more preferably, the invention applies to maize.

Preferably according to the invention, a composition comprising at least cypermethrin and/or natural pyrethrum can be used.

By “furrow” is meant a trench dug in the earth. The characteristics of the furrow are those usually employed in agriculture, and vary depending on the seeds used, which the farmer will know how to adapt.

In furrows, better results will be obtained by incorporating the insecticides regularly at a depth at least greater than 1.5 cm, preferably greater than 2 cm. The width of the area of spread of the composition based on pyrethrum derivatives in the furrow or in the ridge must be at least greater than the largest circumference occupied by the seed.

The method according to the invention can be used against all soil insects or parasites provided they are sensitive to pyrethrum. There may be mentioned for example: click beetles, grey grubs, cockchafer larvae, noctuids, centipede, crane fly, black vine weevil, flies such as the seedling fly and the onion fly or the maize leaf beetle. Preferably the invention relates to the control of click beetles.

As the means of application of the insecticide compositions, it is possible to use applicators for solid formulations or applicators for powder. As applicator of solid formulations it is possible to use granule spreaders or small manually-operated devices for spreading granules such as pushed rotary spreaders for treating smaller areas. Powder applicators are for example devices for manual dusting or tractor-drawn powder-sprinklers. Preferably, application will be carried out with a diffuser, which can be installed at the end of the outlet tubes of a microgranulator.

The invention also relates to a diffuser, which can be fitted to the end of the outlet tubes of microgranulators, particularly on disk drills, also on shoe drills using an installation kit, characterized in that it comprises a cylindrical or truncated body, hollow, open at both ends, one of said ends serving for fixing said diffuser to said outlet tubes of the microgranulator, and the other end being open on an inclined plane, of any shape, preferably of circular or oval shape, integral with said diffuser and having an angle with said diffuser between 35° and 55°, preferably between 40° and 50°, Advantageously said diffuser can further comprise a means of attachment to the outlet tube of the microgranulator. According to a variant, said means for attachment can be a screw positioned perpendicularly to the central axis of the cylinder or truncated cone forming said diffuser and passing through the wall of said cylinder or truncated cone. Such a diffuser can provide precise positioning of the pellets, granules or microgranules of insecticide over the full width of the seed furrow.

Preferably, the uses with ploughing of microgranules into the soil according to the invention can be performed with this diffuser.

The invention also relates to a method of treatment of crops at the time of sowing, characterized in that it uses ploughing into the soil of a composition comprising at least pyrethrum, with the exception of tefluthrin and bifenthrin, in the form of granules or microgranules, preferably in the form of microgranules, preferably according to one of the uses according to the invention.

The invention also relates to the use of a diffuser such as described above for ploughing-in of insecticide, advantageously of solid insecticide. Advantageously according to the invention it will be possible to use the applicant's QUEUE-DE-CARPE DXP™ diffuser.

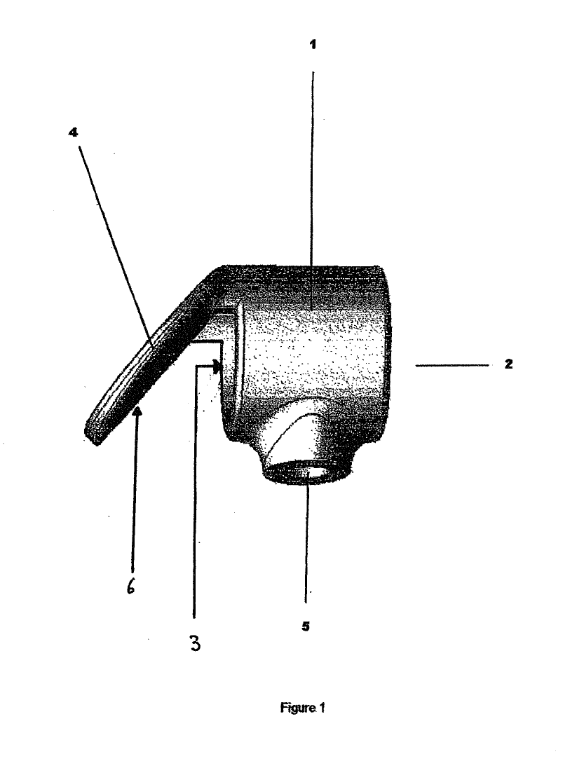

Other characteristics of the invention will become apparent from FIG. 1, which shows a diffuser according to the invention, comprising a cylindrical body (1) having one open end (2), and another end (3) open on an inclined plane (4) of oval shape positioned at 45° relative to the sagittal plane of the cylindrical body. The face (6) of said inclined plane (4), oriented towards end (3) of the diffuser, open on said inclined plane (4), can advantageously not be flat. It can have a ridge positioned on its longitudinal axis, said ridge thus defining, for each of the surfaces located on either side, a slight slope towards the outside of said inclined plane. This arrangement promotes spreading of the composition according to the invention. Said diffuser further comprises a recess (5) that is intended to receive a screw for fixing said diffuser on the end of the outlet tubes of microgranulators.

The invention also relates to a method of treatment of crops at the time of sowing, characterized in that a composition comprising at least pyrethrum, with the exception of tefluthrin and bifenthrin, is used with ploughing into the soil, preferably according to one of the uses described above.

Other characteristics of the invention will become apparent from the examples given below, without the latter constituting any limitation of the invention.

### EXAMPLE 1

### Composition in the Form of Microgranules Based on Cypermethrin Usable According to the Invention

The following mixture is prepared:

Technical cypermethrin (Bayer Bilag (India))

(guaranteed minimum content of 930 g/kg) 73.63%

Methoxypropoxypropanol (Dowanol®) (Dow Chemical Company) 26.37%

Then 1.17% of this mixture is mixed with 98.825% of ground natural calcium carbonate (Granicalcium® 0.35/0.7 SL (OMYA SAS, France)) and a sufficient amount of Rhodamine BSA Extra C (Sigma Aldrich Corp. USA) to obtain, by spraying, 100% of a composition in the form of microgranules that can be used in the method according to the invention.

### EXAMPLE 2

### Control of Click Beetles with Insecticides Localized in Sowing Based on Cypermethrin (Microgranules)

The objective of this study is to evaluate the efficacy of the insecticide cypermethrin used in the form of microgranules against click beetles. The test was conducted at LARREULE (64), a site characterized by a strong presence of click beetles (almost exclusively the species Agriotes sordidus).

Sowing was carried out on Oct. 5, 2007 at a conventional depth (3-4 cm).

The target sowing density is 78000 feet/ha; the variety used is RIXXER®. The soil is clay loam (“touyas” type) with a high level of organic matter (above 4%).

The site is not irrigated.

The meteorological conditions are particularly favourable to the surface activity of click beetles: temperatures are regularly above the seasonal normal and the rainfall, regular to high during the first three months of the maize cycle, promotes the maintenance of moist conditions on the surface of the soil.

Factors Investigated and Methods of Application

In comparison with a reference of carbamate microgranules (CURATER®), a reference of pyrethroid microgranules (tefluthrin) (FORCE 1.5G®) and an untreated control, cypermethrin was tested in the form of microgranules at 12 kg FP/ha.

All of these sets of conditions were applied with a diffuser (with the exception of CURATER®) positioning the granules on the whole surface of the seed furrow. The insecticides used in the tests were:

E1: Control

E2: CURATER® at 12 kg PC/ha (50 g active ingredient/kg) in the seed furrow (SF);

E3: FORCE 1.5 G® at 11.7 kg FP/ha (15 g active ingredient/kg-SF) with diffuser;

E4: Cypermethrin microgranules at 12 kg FP/ha (8 g active ingredient/kg-(SF) with diffuser.

### Conditions for Conducting the Test

Sowing is carried out with a NODET seed drill in 3 rows 0.80 m apart.

The unit plot comprises 3 rows on 20 m. The experiment employs a block device with 4 repetitions. The plots are distributed randomly within each block by means of SILENA software. The central row of 20 m is the subject of the observations.

The plot is weeded and fertilized by the farmer in his usual working conditions (pre-emergence weeding and supply of nitrogen localized between the rows at the 5-6 leaf stage).

After setting up the test, the plots are monitored until the 11-leaf stage, for observations of the vegetation at the start of growing (extent of attack by click beetles), then they are monitored at harvest (proportion of plants with ear).

The timetable of the interventions and observations was as follows:
Sowing: D0
Density at 2 leaves: D0+14
Observation 1: D0+21
Observation 2: D0+27
Observation 3: D0+35
Observation 4: D0+41
Number of ears: D0+90
Harvest: D0+137

Results

1.1. Effect of the Products on Attack at the Start of Vegetation

The level of attack observed on the controls (E1) is high (70.8% at 11 leaves). The reference product (E2) based on carbofuran (CURATER®) shows a poor level of protection (40.6% plants attacked).

Finally, at 11 leaves, cypermethrin in the form of microgranules, applied at 12 kg FP/ha by means of a diffuser (E4: 2.8% plants attacked) shows excellent efficacy.

TABLE 1
Kinetics of attack at the start of vegetation

| | E1 | E2 | E3 | E4 |
|---|---|---|---|---|
| doses | 0 | 12 | 11.7 | 12 |
| Density found at 2-leaf stage | 89.0 | 89.8 | 87.3 | 89.5 |
| % of plants attacked at the | 70.8 | 40.6 | 5.6 | 2.8 |
| 11-leaf stage | | | | |

1.2. Influence of Attacks on the Percentage of Plants Bearing at Least One Harvestable Ear (Ear with More than 70 Seeds):

The results obtained from monitoring the variables at the end of the maize cycle confirm the analysis based on the observations of attacks up to 11 leaves,

TABLE 2
Influence of attacks on the proportion of plants with ears and on
the yield

| | E1 | E2 | E3 | E4 |
|---|---|---|---|---|
| doses | 0 | 12 | 11.7 | 12 |
| % of plants bearing | 70.7 | 82.3 | 95.6 | 96.1 |
| viable ears | | | | |

The results obtained for the variable at the end of the maize cycle (proportion of harvestable ears) confirm the observations made at the start of vegetation. This also confirms, for this criterion, very good efficacy of cypermethrin in the form of microgranules. The efficacy of cypermethrin in the form of microgranules is further confirmed in that, for a similar dose of composition per hectare, the dose of active ingredient per kg of composition is lower than for the other products tested (CURATER® and FORCE 1.5e).

### CONCLUSION

In this test, with heavy infestation by click beetles (70% of plants attacked for the control), cypermethrin in the form of microgranules, applied at 12 kg FP/ha over the full width of the seed furrow by means of a diffuser, displays excellent efficacy against click beetles.

### EXAMPLE 3

### Evaluation of the Efficacy of the Insecticide Cypermethrin at Different Doses Used in the Form of Microgranules Against Click Beetles

The experiment was conducted at LARREULE (64).

This site is characterized by a strong presence of click beetles (almost exclusively the species Agriotes sordidus).

Sowing was carried out on Oct. 5, 2007 at a conventional depth (3-4 cm). The sowing density adopted is 78000 feet/ha; the variety used is RIXXER®.

The soil is clay loam (“touyas” type) with a high level of organic matter (above 4%).

The plot is not irrigated.

The meteorological conditions are particularly favourable to the surface activity of click beetles: temperatures are regularly above the seasonal normal and the rainfall, regular to high during the first three months of the maize cycle, promotes the maintenance of moist conditions on the surface of the soil.

Factors Investigated and Methods of Application

Cypermethrin was tested in the form of microgranules at 12 kg FP/ha in comparison with a reference of carbamate microgranules (CURATER®), a reference of pyrethroid microgranules (tefluthrin) (FORCE 1.5G®) and an untreated control.

All of these sets of conditions were applied with a diffuser (with the exception of CURATER®) positioning the granules or microgranules on the whole surface of the seed furrow.

Products used, methods of application and doses
T1: Control
T2: Reference CURATER® at 12 kg PC/ha (50 g active ingredient/kg) in the seed furrow (SF)
T3: FORCE 1.5 G® at 11.7 kg FP/ha (15 g active ingredient/kg-SF) with diffuser
T4: Cypermethrin microgranules 12 kg FP/ha (8 g active ingredient/kg) (SF) with diffuser

Conditions for Conducting the Test

Sowing is carried out with a NODET seed drill in 3 rows 0.80 m apart.

The unit plot comprises 3 rows on 20 m. The experiment employs a block device with 4 repetitions. The plots are distributed randomly within each block by means of SILENA software. The central row of 20 m is the object of the observations.

The plot is weeded and fertilized by the farmer under his usual working conditions (pre-emergence weeding and supply of nitrogen localized between the rows at the 5-6 leaf stage).

After setting up the test, the plots are monitored until the 11-leaf stage for observations on the vegetation at the start of growing, then at harvest.

The timetable of the interventions and observations is as follows:
Seedlings: D0
Density at 2 leaves: D0+14
Observation 1: D0+21
Observation 2: D0+27
Observation 3: D0+35
Observation 4: D0+41
Number of ears: D0+90
Harvest: D0+137

Results

1.1. Effect of the products on attacks at the start of vegetation

The level of attacks observed on the control (T1) is high (60.7% at 11 leaves). The reference product based on carbofuran (CURATER®, T2) shows a moderate level of protection (21% of plants attacked).

At the 11-leaf stage, the microgranules (T4) based on cypermethrin show an attack rate of 4.3%.

TABLE 3
Kinetics of attack at the start of vegetation

| | product | | | |
|---|---|---|---|---|
| | T1 | T2 | T3 | T4 |
| doses | | 12 | 11.7 | 12.0 |
| % of plants attacked at the 11-leaf stage | 60.7 | 21.0 | 8.9 | 4.3 |

The results show very good protection obtained with cypermethrin in the form of microgranules applied with a diffuser at a dose of 12 kg FP/ha.

1.2. Influence of Attacks on the Proportion of Plants Bearing at Least One Harvestable Ear (Ear with More than 70 Seeds)

TABLE 4
Influence of attacks on yield

| | T1 | T2 | T3 | T4 |
|---|---|---|---|---|
| doses | | 12 | 11.7 | 12.0 |
| % of plants bearing | 60.0 | 83.6 | 95.2 | 97.5 |
| viable ears | | | | |

The results obtained for the variable at the end of the maize cycle (% harvestable ears) confirm the observations of attacks up to the 11-leaf stage.

In this test with heavy infestation by click beetles (60% of plants attacked for the control), cypermethrin in the form of microgranules applied over the full width of the seed furrow by means of a diffuser, at dose of 12 kg FP/ha, shows excellent efficacy against click beetles. The efficacy of cypermethrin in the form of microgranules is further confirmed in that, for a similar dose of composition per hectare, the dose of active ingredient per kg of composition is lower than for the other products tested (CURATER® and FORCE 1.5G®).

1. A method of controlling soil insects comprising ploughing into the soil a composition comprising at least pyrethrum, with the exception of tefluthrin and bifenthrin, in the form of granules or microgranules, preferably in the form of microgranules.

2. The method according to claim 1 for protection of a furrow crop against soil insects comprising the following steps:
a) making a furrow in the soil;
b) placing seed of the furrow crop on the earth at the bottom of the seed furrow formed by the furrow,
c) then spreading said composition over the full width of the seed furrow in which the seed was placed; and
d) closing the furrow with the earth prepared in step c).

3. The method according to claim 1, for protection of a ridge crop against soil insects, comprising the following steps:
a) placing seed of the ridge crop on the earth;
b) then spreading said composition on an area equivalent to the largest circumference occupied by the seed; and
c) forming a ridge by covering the seed placed in a) with the earth prepared in step b).

4-5. (canceled)

6. The method according to claim 2, wherein the seed is a seed of maize, colza, sorghum, sunflower, potato, beetroot, carrot, cereals, cabbages, beans, melon, tomato, ornamental plants or even tobacco, preferably maize seed.

7. The method according to claim 1, wherein the composition comprises cypermethrin and/or natural pyrethrum.

8. The method according to claim 1, wherein the soil insects are selected from the group consisting of click beetle, grey grub, cockchafer larva, noctuids, centipede, crane fly, black vine weevil (Otiorhynchus sulcatus), flies such as seedling fly and onion fly and the maize leaf beetle, preferably click beetle.

9. The method according to claim 1, wherein the composition is used at a dose in the range from 2 to 20 kg/ha, preferably from 9 to 15 kg/ha, and very preferably of 12 kg/ha.

10. The method according to claim 1, wherein pyrethrum is present in the composition in a proportion corresponding to 2 to 12 grams of active ingredient per kilogram of composition, preferably from 6 to 10 g/kg, and very preferably from 7 to 9 g/kg.

11. A diffuser comprising a hollow cylindrical or truncated body, open at both ends, one of said ends serving for fixing said diffuser to the outlet tubes of a microgranulator, and the other end being open on an inclined plane, of any shape, preferably of circular or oval shape, integral with said diffuser and making with said diffuser an angle between 35° and 55°, preferably between 40° and 50°.

12. The diffuser according to claim 11, wherein it comprises a means of attachment to the outlet tube of the microgranulator, advantageously a screw positioned perpendicularly to the central axis of the cylinder or truncated cone forming said diffuser and passing through the wall of said cylinder or truncated cone.

13. The method according to claim 1, wherein the composition is ploughed into the soil by means of a diffuser comprising a hollow cylindrical or truncated body, open at both ends, one of said ends serving for fixing said diffuser to the outlet tubes of a microgranulator, and the other end being open on an inclined plane, of any shape, preferably of circular or oval shape, integral with said diffuser and making with said diffuser an angle between 35° and 55°, preferably between 40° and 50°.

14. A method of treating crops at the time of sowing, wherein a composition is used, with ploughing into the soil, said composition comprising at least pyrethrum in the form of granules or microgranules, preferably in the form of microgranules.

15. (canceled)

16. The method according to claim 1 for protection of a furrow crop against soil insects comprising the following steps:
a) making a furrow in the soil;
b) spreading said composition over the full width of the seed furrow;
c) placing seed of the furrow crop on the earth at the bottom of the seed furrow formed by the furrow, and
d) closing the furrow with the earth prepared in step b).

17. The method according to claim 1, for protection of a ridge crop against soil insects, comprising the following steps:
a) spreading said composition on an area equivalent to the largest circumference to be occupied by seed; and
b) placing seed of the ridge crop on the earth;
c) forming a ridge by covering the seed placed in b) with the earth prepared in step a).

18. The method according to claim 1 for protection of a furrow crop against soil insects comprising the following steps:
a) making a furrow in the soil;
b) simultaneously positioning seed of the furrow crop on the earth at the bottom of the seed furrow formed by the furrow and spreading said composition over the full width of the seed furrow; and
c) closing the furrow with the earth prepared in step b).

19. The method according to claim 1, for protection of a ridge crop against soil insects, comprising the following steps:
a) simultaneously positioning seed of the ridge crop on the earth and spreading said composition on an area equivalent to the largest circumference to be occupied by seed; and
b) forming a ridge covering the seed with the earth prepared in step a).
